(19) United States
(12) Patent Application Publication
(10) Pub. No.: US 2026/0174998 A1
(43) Pub. Date: Jun. 25, 2026

(54) MEDICAL DEVICE WITH COLOR CHANGING HYDROPHILIC COATING

(71) Applicant: Boston Scientific Scimed, Inc., Maple Grove, MN (US)

(72) Inventors: Judith Tiferes Wang, Shoreview, MN (US); Qian Liu, Plymouth, MN (US); Kent Jeffrey Grove, Sartell, MN (US); Brian R. Reynolds, Ramsey, MN (US)

(73) Assignee: Boston Scientific Scimed, Inc., Maple Grove, MN (US)

(21) Appl. No.: 19/424,621

(22) Filed: Dec. 18, 2025

Related U.S. Application Data
(60) Provisional application No. 63/736,362, filed on Dec. 19, 2024.

(51) Int. Cl. A61M 25/06; A61M 25/00

(52) U.S. Cl. CPC A61M 25/0662; A61M 25/002; A61M 25/0045; A61M 2025/0046; A61M 2205/0222; A61M 2205/0227; A61M 2205/0238; A61M 2205/584; A61M 2209/06

(57) ABSTRACT

A medical device includes a device body having an outer surface with visual indicia and a coating extending over at least part of the outer surface. The coating may include a hydrophilic material with a hydrochromic additive that appears opaque when dry and transparent when wet, allowing the visual indicia to be revealed only upon activation with moisture. The visual indicia may include colors or alphanumeric characters that become visible when the coating is wetted. This color-changing mechanism provides users with a clear visual indication of proper coating activation.

### CROSS REFERENCE TO RELATED APPLICATIONS

This application claims the benefit of U.S. Provisional Patent Application Ser. No. 63/736,362 filed on Dec. 19, 2024, the disclosure of which is incorporated herein by reference.

### TECHNICAL FIELD

The present disclosure pertains to medical devices, and methods for manufacturing and using medical devices. More particularly, the present disclosure pertains to medical devices that change color to indicate whether a hydrophilic coating on the medical device has been hydrated.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a medical device that includes a medical device body having an outer surface with visual indicia on the outer surface, and a coating that extends over at least part of the outer surface that is adapted to hide the visual indicia when dry and reveal them when wet.

Alternatively or additionally, the coating may be opaque when dry and transparent when wet.

Alternatively or additionally, the visual indicia may have a color that is different from the color of the coating when dry.

Alternatively or additionally, the visual indicia may include one or more alphanumeric characters.

Alternatively or additionally, the coating may be a hydrophilic coating.

Alternatively or additionally, the coating may include a hydrochromic additive.

Alternatively or additionally, the coating may be a hydrophilic coating with a hydrochromic additive mixed into the hydrophilic coating.

Alternatively or additionally, the coating may have a first layer with a hydrophilic coating and a second layer disposed over the first layer, where the second layer includes a hydrochromic additive.

Alternatively or additionally, the second layer may be adapted to allow water to pass through the second layer and reach the first layer.

In an example, a medical assembly may include a packaging element defining a package interior and the medical device described above disposed within the package interior.

Alternatively or additionally, flushing the package interior with an aqueous solution may wet the coating.

In an example, a device adapted to be used as part of a medical procedure may include a device body having an outer surface with a visual indicator disposed on the outer surface, and a hydrophilic coating extending over at least part of the outer surface, where the coating appears white when dry and transparent when wet.

Alternatively or additionally, the hydrophilic coating may include a hydrochromic material and the visual indicator may have a color that is visually perceptible as not being white.

In an example, an introducer sheath may include an elongate tubular member having an outer surface, a visual indicator disposed on the outer surface, and a hydrophilic coating extending over the outer surface, where the hydrophilic coating is adapted to be opaque when dry and transparent when wet.

Alternatively or additionally, the visual indicator may include one or more alphanumeric characters and the hydrophilic coating may include a hydrochromic additive.

In an example, a medical device may include a medical device body having an outer surface with visual indicia disposed on the outer surface, and a coating extending over at least part of the outer surface that is adapted to hide the visual indicia when dry and reveal them when wet.

Alternatively or additionally, the coating may be opaque when dry and transparent when wet.

Alternatively or additionally, the visual indicia may have a color that is different from the color of the coating when dry.

Alternatively or additionally, the visual indicia may include one or more alphanumeric characters.

Alternatively or additionally, the coating may be a hydrophilic coating.

Alternatively or additionally, the coating may include a hydrochromic additive.

Alternatively or additionally, the coating may be a hydrophilic coating with a hydrochromic additive mixed into the hydrophilic coating.

Alternatively or additionally, the coating may have a first layer with a hydrophilic coating and a second layer disposed over the first layer, where the second layer includes a hydrochromic additive.

Alternatively or additionally, the second layer may be adapted to allow water to pass through the second layer and reach the first layer.

In an example, a medical assembly may include a packaging element defining a package interior and the medical device described above disposed within the package interior.

Alternatively or additionally, flushing the package interior with an aqueous solution may wet the coating.

In an example, a device adapted to be used as part of a medical procedure may include a device body having an outer surface with a visual indicator disposed on the outer surface, and a hydrophilic coating extending over at least part of the outer surface, where the coating appears white when dry and transparent when wet.

Alternatively or additionally, the hydrophilic coating may include a hydrochromic material.

Alternatively or additionally, the visual indicator may have a color that is visually perceptible as not being white.

Alternatively or additionally, the visual indicator may include one or more alphanumeric characters.

In an example, an introducer sheath may include an elongate tubular member having an outer surface, a visual indicator disposed on the outer surface, and a hydrophilic coating extending over the outer surface, where the hydrophilic coating is adapted to be opaque when dry and transparent when wet.

Alternatively or additionally, the visual indicator may have a color that is different from the color of the coating when dry.

Alternatively or additionally, the visual indicator may include one or more alphanumeric characters.

Alternatively or additionally, the coating may be a hydrophilic coating.

Alternatively or additionally, the hydrophilic coating may include a hydrochromic additive.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic view of an example medical device having a hydrophilic layer shown dry;

FIG. 2 is a cross-sectional view taken along the line 2-2 of FIG. 1;

FIG. 3 is a schematic view of the example medical device of FIG. 1, having the hydrophilic layer shown wet with one or more visual indicia;

FIG. 4 is a schematic view of the example medical device of FIG. 1 having a hydrophilic layer shown dry;

FIG. 5 is a cross-sectional view taken along the line 5-5 of FIG. 4;

FIG. 6 is a schematic view of the example medical device of FIG. 5 having the hydrophilic layer shown wet with one or more visual indicia; and

FIG. 7 is a schematic view of a medical assembly that includes a medical device that is disposed within a tubular packaging going through a wetting process.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Hydrophilic materials and hydrophilic coatings provide a number of benefits. For example, hydrophilic materials have a low coefficient of friction, which reduces the forces needed to manipulate medical devices. This can help to reduce the risk of damaging blood vessel walls when advancing a hydrophilic medical device through a patient's vasculature. In some cases, there may be a reduced risk of blood clotting, as hydrophilic surfaces allow blood to flow around the device.

Hydrophilic materials may include a variety of different polymers. Hydrophilic materials may include polyurethanes or polyethylenes. In some cases, a hydrophilic material may include UHMWPE (ultra-high molecular weight polyethylene). In some cases, a medical device may have a hydrophilic surface as a result of an outer layer of the medical device being formed of a hydrophilic polymer, including those listed.

In some cases, a medical device may have a hydrophilic surface as a result of a hydrophilic coating that is applied over a structure that is not hydrophilic. Examples of hydrophilic coatings include PVP (polyvinylpyrrolidone), PEG (polyethylene glycol), hyaluronic acid, PAA (polyacrylic acid), PEO (polyethylene oxide), HEMA (hydroxyethyl methacrylate), and cellulose derivatives. Hydrophilic coatings may be applied using a variety of methods, including dip coating, spray coating, plasma treatment, UV curing and chemical vapor deposition.

In some cases, hydrophilic coatings may include one or more hydrochromic additives. Hydrochromic additives are materials that undergo reversible color changes when exposed to water. For example, a material including a hydrochromic additive may appear a first color when dry and appear a second color when wet. As an example, a material including a hydrochromic material or additive may appear white when dry, yet translucent or even transparent when wet. Describing a surface or material as wet may be defined as the surface or material being covered or saturated with a liquid. In some cases, describing a surface or material as wet may be defined as the surface or material being covered or saturated with water.

The basic mechanism by which hydrochromic materials change color may involve either chemical or physical interactions with water molecules. A variety of chemical interactions are possible. An example of chemical interactions includes ion hydration. In some cases, water molecules may coordinate with particular metal ions (e.g., Co²⁺ in CoCl₂). Changes in ion coordination may alter light absorption properties, resulting in visible color changes. In some cases, molecular conformational changes include water triggering structural changes in chromophore molecules. Altered molecular structure absorb different wavelengths of light, thereby creating visible color changes. Physical mechanisms include structural changes. As an example, water absorption may cause swelling or reorganization. Resultant changes in material structure can affect light interaction, which can result in altered optical properties. In some cases, interface effects include water modifying the surface properties of a surface, including changing the refractive index. Changing the refractive index can impact light reflection and/or light transmission.

An example of a hydrochromic material is cobalt chloride, which changes from blue to pink when exposed to moisture. Another example is copper chloride, which changes from blue-green to yellow with hydration. A number of hydrochromic dyes include spiropyrans, leuco dues and crystal violet lactone. Some polymer-based materials qualify as hydrochromic materials. Examples include PVA (polyvinyl alcohol) with chromogenic additives. Color-changing hydrogels and smart polymer films are additional examples. Some metal complex-based materials function as hydrochromic materials. Metal complex-based materials may include nickel complexes, iron compounds and ruthenium-based materials, for example. There are some natural hydrochromic materials, such as some plant-derived pigments, bacterial pigments, and modified cellulose with chromogenic properties. Some composite materials function as hydrochromic materials, such as polymer-dye combinations, nanoparticle-embedded matrices and hydrogel-chromophore systems.

Additional examples include dye types such as benzofuran compounds, lactone compounds, fluorine compounds, and aniline compounds, with the dye content ranging from 0.5 to 35 percent by weight. In some cases, the dye content ranges from 1 to 10 percent by weight. In some cases, the dye content ranges from 3 to 5 percent by weight. The hydrochromic composition may include pigments at 0.01 to 10 percent by weight, a resin mixture of ketonic and cellulosic compounds at 5 to 40 percent by weight, and solvents at 30 to 80 percent by weight. These materials demonstrate key properties including color change upon water contact, excellent heat and humidity resistance, good light fastness, and improved viscosity and printing workability.

In some instances, a medical device includes a medical device body that has an outer surface with one or more visual indicia disposed on the outer surface. A coating extends over at least part of the outer surface and is adapted to hide the visual indicia when the coating is dry and to reveal the visual indicia when the coating is wet. Accordingly, the visual indicia becoming visible indicates that the coating has been wetted. In some cases, the coating may include a hydrophilic material that becomes more hydrophilic when wetted.

In some cases, the coating may be opaque when dry and transparent when wet. In some cases, the visual indicia include a color that is different from a color of the coating when the coating is dry. In some cases, the visual indicia may include one or more alphanumeric characters. The coating may include a hydrophilic coating. In some cases, the coating may include a hydrochromic additive. In some cases, the coating may include a hydrophilic coating having a hydrochromic additive mixed into the hydrophilic coating. In some cases, the coating may include a first layer that includes a hydrophilic coating and a second layer including a hydrochromic additive that is disposed over the first layer. In some cases, the second layer may be adapted to allow water to pass through the second layer and reach the first layer.

In some instances, a medical assembly may include a packaging element that defines a package interior. A medical device may be disposed within the package interior. The medical device includes a medical device body that has an outer surface with one or more visual indicia disposed on the outer surface. A coating extends over at least part of the outer surface and is adapted to hide the visual indicia when the coating is dry and to reveal the visual indicia when the coating is wet. In some cases, flushing the package interior with an aqueous solution wets the coating.

In some instances, a device is adapted to be used as part of a medical procedure. The device includes a device body having an outer surface. The outer surface includes a visual indicator that is disposed on the outer surface. A hydrophilic coating extends over at least part of the outer surface. The coating appears white when dry and appears transparent when wet. In some cases, the hydrophilic coating may include a hydrochromic material. In some cases, the visual indicator may include a color that is visually perceptible as not being white. In some cases, the visual indicator may include one or more alphanumeric characters.

In some instances, an introducer sheath includes an elongate tubular member having an outer surface. A visual indicator is disposed on the outer surface. A hydrophilic coating extends over the outer surface. The hydrophilic coating is adapted to be opaque when dry and transparent when wet. In some cases, the visual indicator includes a color that is different from a color of the coating when the coating is dry. In some cases, the visual indicator may include one or more alphanumeric characters. In some cases, the coating may include a hydrophilic coating. In some cases, the hydrophilic coating may include a hydrochromic additive.

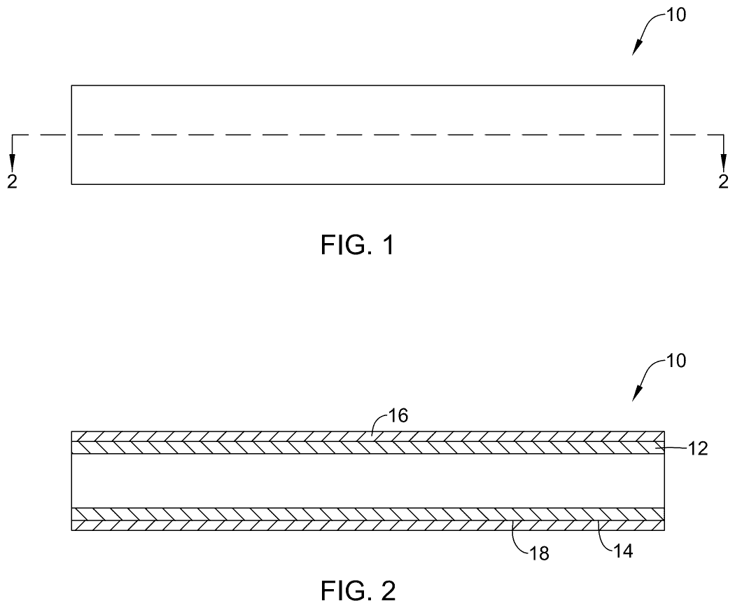

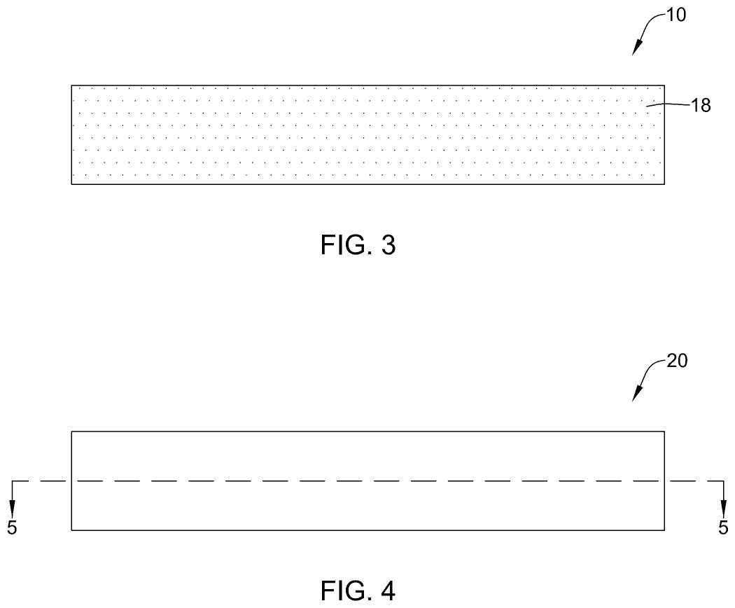

A number of medical devices may benefit from including a hydrophilic outer surface that reduces friction. Examples include catheters, guide catheters, introducer sheaths, and others. FIG. 1 is a schematic view of an example medical device 10 that may represent any of a variety of different medical devices, including the aforementioned catheters, guide catheters and introducer sheathes. FIG. 2 is a cross-sectional view of the example medical device 10, taken along the line 2-2 of FIG. 1. FIG. 3 is a schematic view of an example medical device having a hydrophilic layer shown wet with one or more visual indicia. The medical device 10 includes a medical device body 12 that has an outer surface 14. In some cases, one or more visual indicia 18 may be disposed on the outer surface 14. In the case of the medical device 10, the one or more visual indicia 18 includes the outer surface 14 including a color, as shown in FIG. 3. In some cases, the one or more visual indicia 18 may include alphanumeric characters, pictures, or other images. In some cases, the one or more visual indicia 18 may include simple instructions, such as an arrow indicating an insertion direction. Another example of simple instructions that may be included in the one or more visual indicia may include “THIS SIDE UP”, or “THIS END UP”.

The medical device 10 includes a coating 16 that extends over at least part of the outer surface 14. The coating 16 is adapted to have a first color when the coating 16 is dry and to have a second color that is different from the first color, or to be translucent or even transparent when the coating 16 is wet. As an example, the coating 16 may appear white when dry, as seen for example in FIG. 1. The coating 16 may become transparent when wet, which allows the one or more visual indicia to become visible. FIG. 3 shows the medical device 10 after the coating 16 has become wet. As a result, the coating 16 has become translucent or even transparent, and the color of the one or more visual indicia is now visible. The one or more visual indicia disposed on the outer surface 14 of the medical device body 12, is shown in FIG. 3 as a dot pattern. The one or more visual indicia 18 can now be seen through the coating 16. Accordingly, the one or more visual indicia (the color disposed on the outer surface 14 of the medical device body 12) indicates that the coating 16 has been sufficiently wetted.

In some cases, the coating 16 is a hydrophilic coating. In some cases, the coating 16 may also include one or more hydrochromic materials or additives that allow the coating 16 to change color in accordance with whether the coating 16 is wet or dry. As shown in FIG. 2, the medical device includes a single coating 16 that is disposed over the visual indicia 18 present on the outer surface 14 of the medical device body 12. The single coating 16 may have a thickness that ranges from 3.94×10⁻⁷ inches (10 nanometers) to 3.94×10⁻⁶ inches (100 nanometers). In some cases, the single coating 16 may have a thickness that is as large as a few micrometers. In some cases, as will be discussed with respect to FIGS. 4-6, there may be more than one coating disposed over the outer surface 14 of the medical device body 12. For example, the coating 16 may include a hydrophilic layer and a separate hydrochromic layer. In some cases, when the coating 16 includes two distinct layers, whichever layer is the outermost layer may allow water to move through the outermost layer so that water may also reach the layer under the outermost layer. In some cases, the two distinct layers may each have a thickness that ranges from 3.94×10⁻⁷ inches (10 nanometers) to 3.94×10⁻⁶ inches (100 nanometers). In some cases, the two distinct layers together may have a total thickness that ranges from 3.94×10⁻⁷ inches (10 nanometers) to 3.94×10⁻⁶ inches (100 nanometers).

The coating 16 may be wetted in any of a variety of different processes. In some cases, the medical device 10 may simply be dipped into an aqueous solution, or even distilled water. In some cases, the medical device 10 may be sprayed with water. In some cases, particularly if the medical device 10 is within a package, the package may be flushed with an aqueous solution, or even distilled water, in order to hydrate the coating 16. Hydrating the coating 16 results in the coating 16 becoming more hydrophilic as well as changing color to reveal the one or more visual indicia present underneath the coating 16.

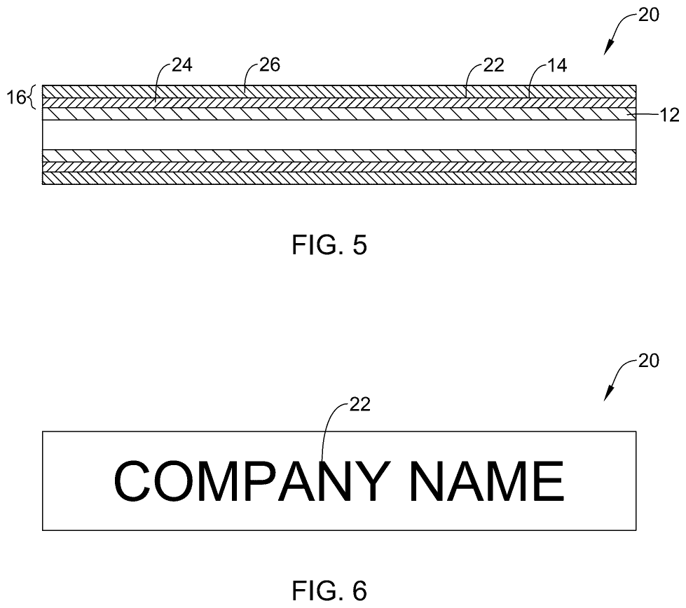

FIG. 4 is a schematic view of an example medical device 20 that may represent any of a variety of different medical devices, including the aforementioned catheters, guide catheters and introducer sheathes. FIG. 5 is a cross-sectional view of the example medical device 20, taken along the line 5-5 of FIG. 4. FIG. 6 is a schematic view of the example medical device having a hydrophilic layer shown wet with one or more visual indicia. The medical device 20 includes a medical device body 12 that has an outer surface 14. In some cases, one or more visual indicia 22 may be disposed on the outer surface 14. In the case of the medical device 20, the one or more visual indicia 22 includes alphanumeric characters that spell out “COMPANY NAME”. While shown generically, it will be appreciated that the particular company that manufactures and/or sells the medical device 20 may display their company name as the one or more visual indicia 22. In some cases, the one or more visual indicia 22 could also spell out a product name, for example. In some cases, the one or more visual indicia 22 could include one or more colors that are different from the dry appearance of the coating 16.

In some cases, the coating 16 may include an inner layer 24 and an outer layer 26. The inner layer 24 and the outer layer 26 may each independently have a thickness that ranges from 3.94×10⁻⁷ inches (10 nanometers) to 3.94×10⁻⁶ inches (100 nanometers). In some cases, the inner layer 24 and the outer layer 26 together may have a combined thickness that ranges from 3.94×10⁻⁷ inches (10 nanometers) to 3.94×10⁻⁶ inches (100 nanometers). In some cases, the inner layer 24, the outer layer 26, or the inner layer 24 and the outer layer 26 together may have a combined thickness that is as large as a few micrometers. In some cases, as will be discussed with respect to FIGS. 4-6, there may be more than one coating disposed over the outer surface 14 of the medical device body 12. In some examples, the inner layer 24 may represent a hydrophilic layer and the outer layer 26 may represent a hydrochromic layer. In some cases, the inner layer 24 may represent a hydrochromic layer and the outer layer 26 may represent a hydrophilic layer. The outer layer 26 may be porous to water such that water impacting the outer layer 26 is able to pass through the outer layer 26 and reach the inner layer 24. That way, in cases when the inner layer 24 is the hydrophilic layer, the hydrophilic layer is able to become wetted and thus become more hydrophilic. In cases when the inner layer 24 is the hydrochromic layer, the hydrochromic layer is able to become wetted and thus undergo a color change, such as changing from white (or another color) to being translucent or even transparent such that the one or more visual indicia 22 may become visible.

The coating 16 may be wetted in any of a variety of different processes. In some cases, the medical device 20 may simply be dipped into an aqueous solution, or even distilled water. In some cases, the medical device 20 may be sprayed with water. In some cases, particularly if the medical device 10 is within a package, the package may be flushed with an aqueous solution, or even distilled water, in order to hydrate the coating 16. In some cases, the coating 16 may be wetted by contacting the coating 16 with a wet towel, a wet sponge, a wet gauze or any other wetted element. Hydrating the coating 16 results in the coating 16 becoming more hydrophilic as well as changing color to reveal the one or more visual indicia present underneath the coating 16.

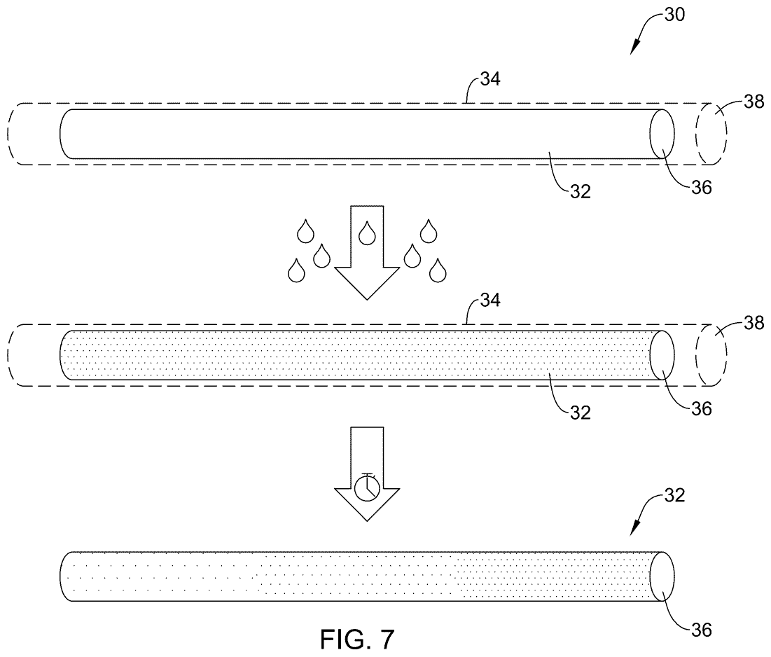

FIG. 7 provides an example of a medical assembly 30 that includes a medical device 32 that is disposed within a tubular packaging 34 going through a wetting process. The medical device 32 may represent an introducer sheath, for example, and defines a device lumen 36. The tubular packaging 34 defines a lumen 38 that accommodates the medical device 32 therein. At the top of FIG. 7, the medical device 32 is dry, as indicated by a first color such as white. In the middle of FIG. 7, the medical device 32 is now wet as a result of flushing the lumen 38 (and hence the inside and outside of the medical device 32) with an aqueous flush. The aqueous flush may be as simple as distilled water, for example. As a result, the outermost layer of the medical device 32 (such as the coating 16 described with respect to FIGS. 1-6) changes color. In some cases, the outermost layer of the medical device 32 becomes transparent to reveal one or more visual indicia (such as the one or more visual indicia 18 shown in FIGS. 2 and 3 or the one or more visual indicia 22 shown in FIGS. 5 and 6). As can be seen, the outermost layer of the medical device 32 has changed colors in the middle of FIG. 7.

Generally, the hydration of the medical device 32 does not last, and in some cases the color changes facilitated by inclusion of a hydrochromic material or additive also are impermanent. The color changes may also fade as the material dries. This can be seen at the bottom of FIG. 7, where the medical device 32, after being wetted, has been removed from the tubular packaging 34. Over time, which may be as short as several minutes, the hydrophilic material and the hydrochromic material or additives also start to dry. As the water evaporates, the hydrochromic material starts returning to its original color, which begins to block visualization of the visual indicia present underneath the hydrochromic material. Accordingly, inclusion of a hydrochromic material provides an easy indication not only of whether the medical device 32 (or the medical device 10 or the medical device 20) has been hydrated, but can also provide an indication of whether the medical device 32 (or the medical device 10 or the medical device 20) remains hydrated. In some cases, the hydrochromic material can also provide a visual indication of how hydrated the medical device 32 (or the medical device 10 or the medical device 20) remains.

The hydrophilic and hydrochromic materials discussed herein may be added to any of a variety of different medical devices. The medical devices receiving hydrophilic and hydrochromic materials and coatings may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

It should be understood that this disclosure is, in many respects, only illustrative. Changes may be made in detail, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device, comprising:
a medical device body having an outer surface, the outer surface including one or more visual indicia disposed on the outer surface;
a coating extending over at least part of the outer surface, the coating adapted to hide the visual indicia when the coating is dry and to reveal the visual indicia when the coating is wet.

2. The medical device of claim 1, wherein the coating is opaque when dry and transparent when wet.

3. The medical device of claim 1, wherein the visual indicia comprises a color that is different from a color of the coating when the coating is dry.

4. The medical device of claim 1, wherein the visual indicia comprises one or more alphanumeric characters.

5. The medical device of claim 1, wherein the coating comprises a hydrophilic coating.

6. The medical device of claim 1, wherein the coating comprises a hydrochromic additive.

7. The medical device of claim 1, wherein the coating comprises a hydrophilic coating with a hydrochromic additive mixed into the hydrophilic coating.

8. The medical device of claim 1, wherein the coating comprises a first layer comprising a hydrophilic coating and a second layer disposed over the first layer, the second layer comprising a hydrochromic additive.

9. The medical device of claim 8, wherein the second layer is adapted to allow water to pass through the second layer and reach the first layer.

10. A medical assembly, comprising:
a packaging element defining a package interior; and
the medical device of claim 1 disposed within the package interior.

11. The medical assembly of claim 10, wherein flushing the package interior with an aqueous solution wets the coating.

12. A device adapted to be used as part of a medical procedure, the device comprising:
a device body having an outer surface, the outer surface including a visual indicator disposed on the outer surface; and
a hydrophilic coating extending over at least part of the outer surface, the coating appearing white when dry and transparent when wet.

13. The device of claim 12, wherein the hydrophilic coating comprises a hydrochromic material.

14. The device of claim 12, wherein the visual indicator comprises a color that is visually perceptible as not being white.

15. The device of claim 12, wherein the visual indicator comprises one or more alphanumeric characters.

16. An introducer sheath, comprising:
an elongate tubular member having an outer surface;
a visual indicator disposed on the outer surface;
a hydrophilic coating extending over the outer surface, the hydrophilic coating adapted to be opaque when dry and transparent when wet.

17. The introducer sheath of claim 16, wherein the visual indicator comprises a color that is different from a color of the coating when the coating is dry.

18. The introducer sheath of claim 16, wherein the visual indicator comprises one or more alphanumeric characters.

19. The introducer sheath of claim 16, wherein the coating comprises a hydrophilic coating.

20. The introducer sheath of claim 16, wherein the hydrophilic coating comprises a hydrochromic additive.
